(19) United States
(12) Patent Application Publication
(10) Pub. No.: US 2004/0236284 A1
(43) Pub. Date: Nov. 25, 2004

(54) Needle hiding assembly for a medication injector

(76) Inventors: Shannon Marie-Lynn Hoste, Westerville, OH (US); Scott Forster Percy, Carmel, IN (US); Michael Scott Ulrich, Columbus, OH (US); Grant Richard Wilson, Columbus, OH (US)

Correspondence Address: ELI LILLY AND COMPANY, PATENT DIVISION, P.O. BOX 6288, INDIANAPOLIS, IN, 46206-6288, US

(21) Appl. No.: 10/479,112

(22) PCT Filed: Jun. 4, 2002
(86) PCT No.: PCT/US02/22280

Related U.S. Application Data
(60) Provisional application No. 60/297,053, filed on Jun. 8, 2001.

(51) Int. Cl. A61M 5/32

(52) U.S. Cl. 604/198

(57) ABSTRACT

An assembly mountable to an injector, such as an injection pen, to hide the needle of the injector from view. The assembly includes a retractable cover that is slidably mounted on a base sleeve that is releaseably connected to the injector. A spring between the cover and the base sleeve biases the cover toward a forward position at which it surrounds and hides the axially fixed needle from view.X-14348

### BACKGROUND OF THE INVENTION

The present invention pertains to medication injector devices such as injection pens, and, in particular, to an assembly that covers the needle of an injection pen when not in use.

Patients suffering from diseases such as diabetes frequently must inject themselves with medication, such as insulin solutions. To permit a person to conveniently and accurately self-administer proper doses of medicine, a variety of devices broadly known as injection pens have been developed.

One problem with needled injection pens is that some users, especially children, may be traumatized by the sight of the injection needle before and during the process of injection. Some devices, such as disclosed in U.S. Pat. No. 5,873,856, which are designed to control needle penetration depth, have a sliding cover which hides the needle when not being used, and which retracts a selected distance by contact with the user's skin at the injection site during use. While useful to hide the needle, such devices are not without their shortcomings. For one thing, in order to achieve the penetration depth controlling feature, the devices are relatively complicated in design. In addition, and during such times as the mounting of the device to an injection pen, inadvertent contact which moves an exposed sliding cover can result in an unwanted needle stick.

Thus, it would be desirable to provide an apparatus that overcomes these and other shortcomings of the prior art.

### BRIEF SUMMARY OF THE INVENTION

In one form thereof, the present invention provides a needle hiding assembly for a portable injector having a needle forwardly axially protruding from a forward end of the injector, the needle being in communication with a medicine filled cartridge of the injector. The needle hiding assembly includes a base sleeve releaseably connected to the injector, a needle cover slidably retained on the base sleeve to be axially movable between forward and rearward positions, wherein the needle cover when in the forward position surrounds at least a forward part of the needle, the needle cover including a forward surface that defines a needle accommodating opening through which the needle extends when the needle cover is shifted from the forward position to the rearward position, wherein a length of the needle that projects forward beyond the forward surface is exposed for injection when the needle cover is disposed in the rearward position, a biasing element acting between the base sleeve and the needle cover to bias the needle cover from the rearward position toward the forward position, and a cap releaseably connectable to the base sleeve and defining an interior hollow that accommodates the needle cover, wherein the cap and needle cover are configured such that connecting the cap to the base sleeve shifts the needle cover from the forward position toward the rearward position against a returning force provided by the biasing element.

In another form thereof, the present invention provides a needle hiding assembly for an injection pen having a needle forwardly axially protruding from a forward end of the injection pen, the needle being in communication with a medicine filled cartridge of the injection pen. The needle hiding assembly includes a base sleeve releaseably connected to the injection pen, a needle cover slidably retained on the base sleeve to be axially movable between forward and rearward positions, wherein the needle cover when in the forward position surrounds at least a forward part of the needle, the needle cover including a forward surface that defines a needle accommodating opening through which the needle extends when the needle cover is shifted from the forward position to the rearward position, wherein a length of the needle that projects forward beyond the forward surface is exposed for injection when the needle cover is disposed in the rearward position, a biasing element acting between the base sleeve and the needle cover to bias the needle cover from the rearward position toward the forward position, and wherein the forward surface of the needle cover has a first region and a second region, wherein the first region encircles the needle accommodating opening and is located between the needle accommodating opening and the second region, and wherein the first region is rearwardly recessed from the second region to define a concavity for increased needle length exposure.

One advantage of the present invention is that a needle hiding assembly mountable to an injection pen is provided which has a slidable cover that hides the pen needle from view during non-use, and which retracts when contacted with the skin during use to administer medicine contained in the pen.

Another advantage of the inventive needle hiding assembly is that a cap is included which fits over the retractable cover when the injection pen is not being used to reduce the likelihood of accidental needle sticking.

Another advantage of the inventive needle hiding assembly is that its cap retracts the retractable cover when installed there over so as to minimize the cap profile and improve the overall appearance of the pen and improve the portability of the pen if being carried with the user between uses.

Still another advantage of the inventive needle hiding assembly is that the surface of the retractable cover which contacts the patient during use has a central concavity to promote needle penetration by exposing the entire length of the needle when the cover is in the retracted position, and to minimize the size and profile of the assembly.

### BRIEF DESCRIPTION OF THE DRAWINGS

The above-mentioned and other advantages and objects of this invention, and the manner of attaining them, will become more apparent, and the invention itself will be better understood, by reference to the following description of embodiments of the invention taking in conjunction with the accompanying drawings, wherein:

FIG. 1 is a front view of a medicine injection pen equipped with one form of a needle hiding assembly of the present invention;

FIG. 2 is a front view of the medicine injection pen with needle hiding assembly of FIG. 1 after removal of the outer cap of the assembly;

FIG. 3 is a diagrammatic, cross-sectional front view of the needle hiding assembly of FIG. 1, wherein portions of the injection pen around which the assembly is installed are abstractly shown;

FIG. 4 is diagrammatic, cross-sectional view of the needle hiding assembly of FIG. 2, taken through a plane offset forty-five degrees from the plane of the view of FIG. 3, wherein portions of the injection pen around which the assembly is installed are abstractly shown;

FIG. 5 is an exploded perspective view of the needle hiding assembly of FIG. 1 prior to its mounting over the injection pen;

FIG. 6 is a cross-sectional front view of the exploded needle hiding assembly of FIG. 5;

FIG. 7 is another cross-sectional view of the slidable cover shown separate from the remainder of the needle hiding assembly of FIG. 6; and

FIG. 8 is a cross-sectional view of the saddle section of the assembly base to which the cover is slidably mountable shown separate from the remainder of the needle hiding assembly of FIG. 6.

Corresponding reference characters indicate corresponding parts throughout the several views. Although the drawings represent one embodiment of the present invention, the drawings are not necessarily to scale, and certain features may be exaggerated or omitted in some of the drawings in order to better illustrate and explain the present invention.

### DETAILED DESCRIPTION OF THE INVENTION

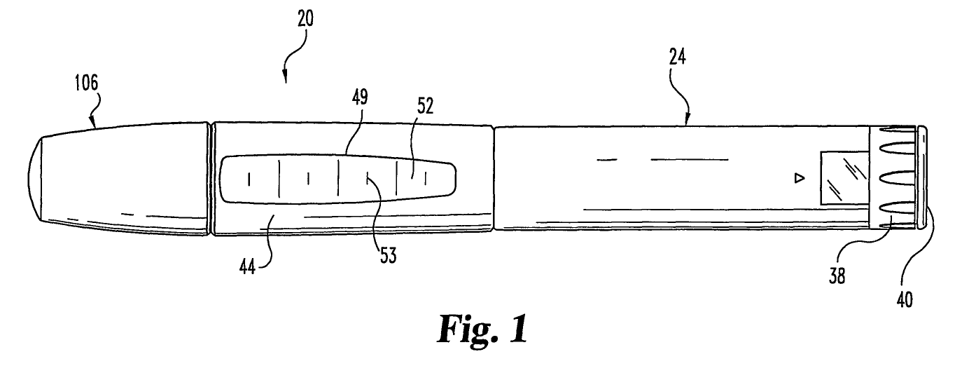

FIG. 1 illustrates an exemplary embodiment of the needle hiding assembly of the present invention mounted to a suitable portable medicine injector. The needle hiding assembly, generally designated 20, can find beneficial application with both disposable and reusable injectors which are variously configured, and the specifics of the injector disclosed herein are illustrative and not intended to be limiting.

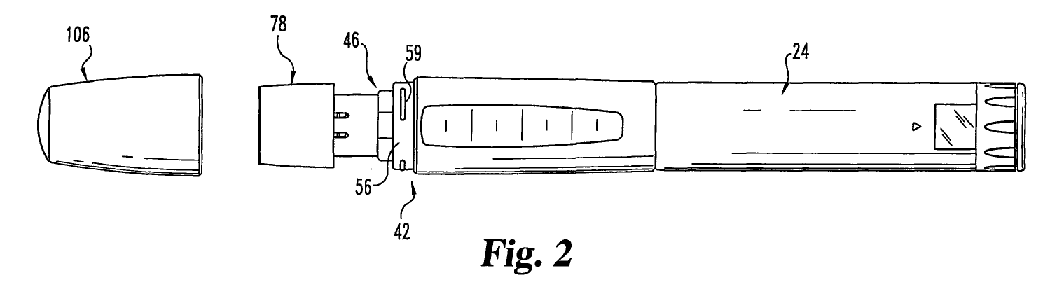

The shown injector is a pen-type injection device and includes a reusable rearward base 24 to which is mounted, such as via a threaded engagement, a cartridge assembly 26 that in FIGS. 1 and 2 is protectively surrounded by needle hiding assembly 20. Cartridge assembly 26 is designed to be removed as a unit and discarded when its medication is exhausted, and then replaced with a new cartridge assembly. Other suitable cartridge assemblies, such as reusable retainers in which are loaded disposable medication cartridges, are also known.

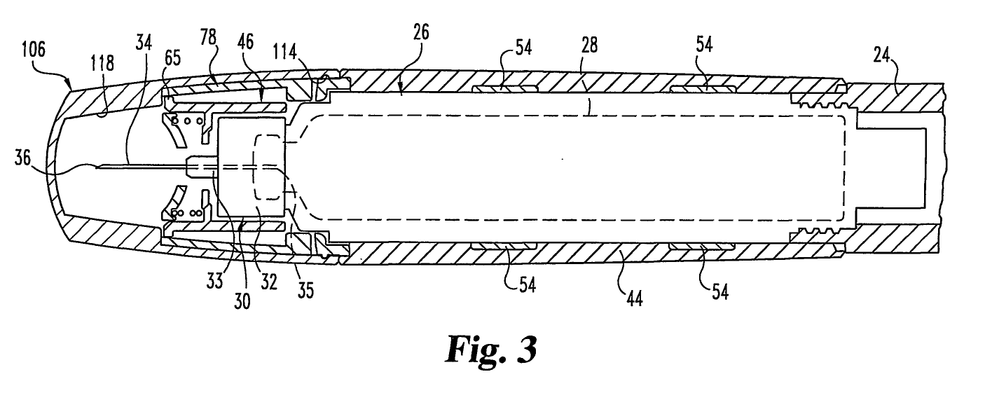

As further shown in FIG. 3, cartridge assembly 26 holds a cartridge 28 that defines an internal volume filled with a multi-dose quantity of medicine, such as insulin or human growth hormone. The cartridge assembly is preferably made with transparent components which allow the contents of the cartridge to be visible. A plunger seals the rearward open end of the cartridge internal volume and is slidable forward during pen operation to force medicine from the cartridge. An injection needle assembly, generally designated 30, includes a hub 32 with a needle retaining projecting portion 33, and a needle or cannula 34. When injection needle assembly 30 is mounted to the forward end of cartridge assembly 26 as shown in FIG. 3, such as via a threaded or snap fit engagement of hub 32 with the cartridge assembly, the rearward tip 35 of needle 34 pierces a septum that otherwise seals the forward open end of the cartridge internal volume so as to communicate with the cartridge interior, and the forward end 36 of needle 34 projects axially beyond the forward end of the injector pen for insertion into a user's skin during injecting use of the pen.

An exposed knob 38 with rotatable button 40 thereon at the rearward or distal end of pen base 24 is operably connected with other components of the dose setting and injecting mechanisms of the shown pen. During the dose setting process by which the quantity of medication to be delivered by operation of the pen is selected by the user, knob 38 is rotatable to set the dose, and when knob 38 is so rotated to increase the selected dose the knob 38 and button 40 translate out of pen base 24 from the axial position shown in FIG. 1. During the dose injecting process which occurs after the dose setting process, when a plunging force is applied to button 40, which rotates freely relative to knob 38, button 40 and knob 38 are designed to be shifted to the left, and back to the axial position shown in FIG. 1, to cause the injecting mechanism components housed within the pen base to operate to slide the cartridge plunger forward to force medicine to be expelled from cartridge 28 through needle 34.

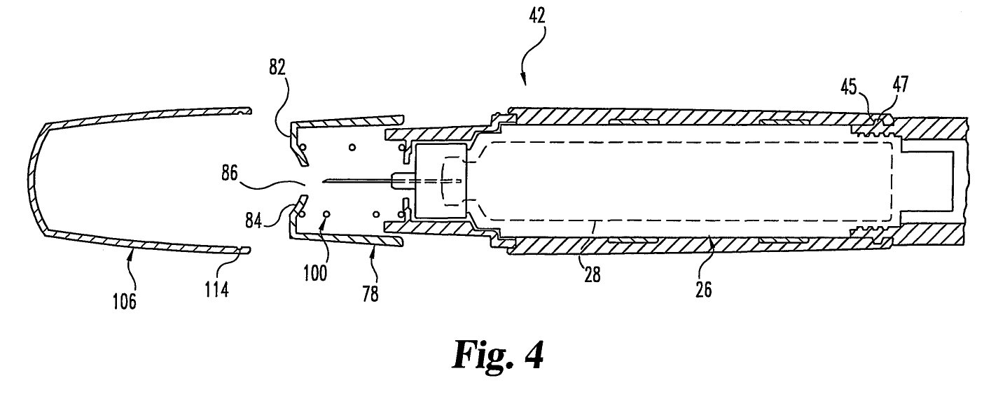

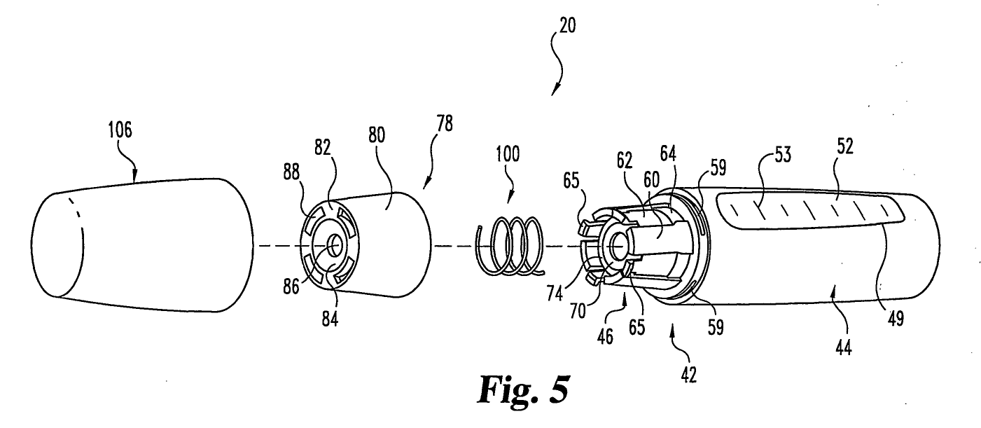

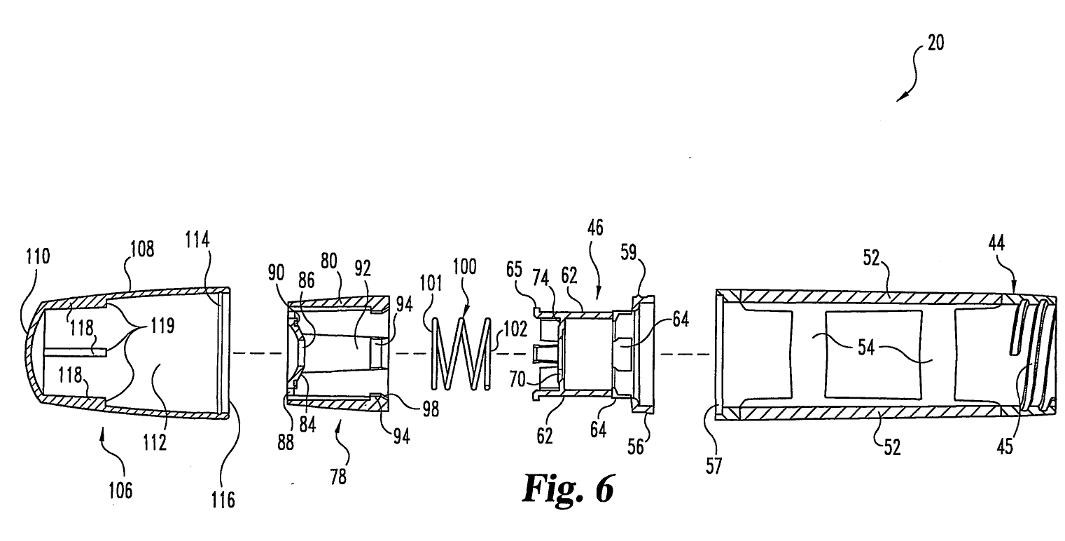

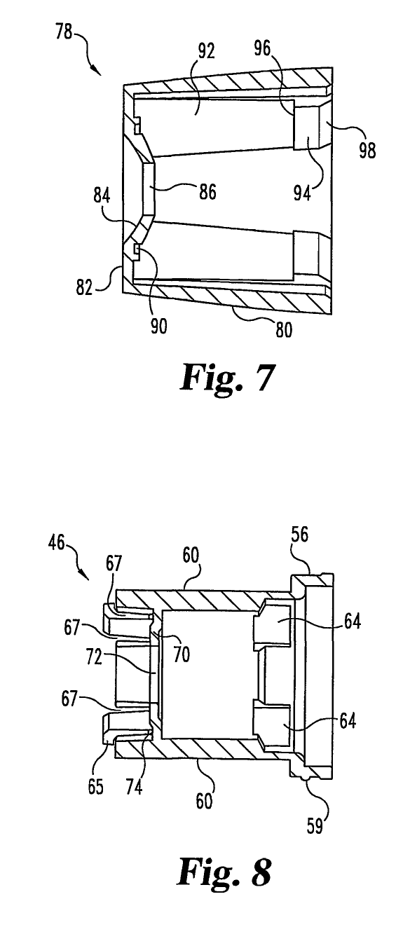

Needle hiding assembly 20 includes a tubular or sleeve-shaped base 42 which fits over cartridge assembly 26. Base 42 extends over substantially the entire length of the cartridge assembly 26, and in the preferred embodiment covers all of the cartridge assembly length not inserted within pen base 24, thereby providing cartridge 28 with additional protection from damage. A generally cylindrical body section 44 of base 42 is made of an opaque material such as plastic. Body section 44 has a rearward end which is internally threaded at 45 to mate with external threading 47 provided on the pen base housing that is abstractly shown in FIGS. 3 and 4 and to which is mounted cartridge assembly 26. Threads 45 provide for an intuitive, releaseable connection of assembly 20 to the injection pen such that base 42 is axially fixed relative to the pen. Other known forms of connection, such as a bayonet fitting or a snap fit, may alternatively be used in place of the shown threads.

A pair of longitudinally aligned windows 49 are diametrically located in body section 44 to allow cartridge visibility. An insert assembled with body section 44 using a two-shot molding process during manufacture is made of clear plastic and includes two portions 52 which fill or cover windows 49. Markings 53 on portions 52 aid a user in gauging how much medicine remains in the contained cartridge. Window covering portions 52 are spanned on each side by arcuate bars 54 that fit within channels formed in the interior surface of base body section 44.

A saddle section 46 of base 42 serves to mount the slidable cover of needle hiding assembly 20. In the shown embodiment in which the saddle section 46 and body section 44 are separately molded and then assembled together, a rearward region of annular portion 56 of saddle section 46 fits within a circumferential notch 57 of body section 44. Ultrasonic welding preferably is used to fixedly secure together saddle section 46 and body section 44, but other forms of securement, such as adhesives, may be used. In alternate embodiments, base 42 could be otherwise configured, and could be formed or molded as a single piece.

A series of angularly spaced and radially projecting ribs 59 ring the forward region of annular portion 56. In the shown embodiment, three circumferentially aligned ribs 59 centered one hundred twenty degrees apart are employed. Differently shaped or configured modules suitable for releaseable connection of the cap may be substituted for ribs 59.

The exterior surface of the region of saddle section 46 which projects forward of annular portion 56 is stepped in from portion 56 and includes four generally rectangular areas 60 and four radially inwardly recessed areas 62 which are sequentially arranged around the saddle section circumference. The space between adjacent areas 60 and radially outward of recessed area 62 forms a channel in which slide stop tabs 94 of cover 78. Openings 64 extend through saddle section 46 at the rearward end of each recessed area 62 to accommodate stop tabs 94 during cover retraction.

At its forward edge which protrudes forward of areas 60, each recessed area 62 is equipped with an outwardly projecting tang 65 used to retain assembly cover 78 to base 42. Longitudinally aligned notches 67 in the recessed areas 62 at locations immediately adjacent areas 60 result in the tanged recessed areas being elastically bendable slightly inward during initial manufacturing assembly of the cover 78 to saddle section 46.

Spanning the otherwise open interior of saddle section 46 is an annular shoulder 70 that defines a central opening 72 for needle passage. A stepping of shoulder 70 forms a ring-shaped groove 74 in which is seated the rearward end of compression spring 100.

A retractable needle hiding cover 78 made of plastic is slidably mounted to saddle section 46. Cover 78 includes a tubular main body 80 which has a curved but generally frustroconical exterior surface. The forward end face of cover 78 which extends inward from main body 80 has an annular, planar outer region 82 and an annular inner region 84.

Inner region 84 defines a circular opening 86 for needle passage when cover 78 is slid rearward to expose the fixed needle. Outer region 82 is designed to abut a user's epidermis during use. In a preferred embodiment, opening 86 has a diameter of about 0.170 inch, and inner region 84 has an outer diameter of about 0.390 inch. Outer region 82 has an outer diameter of about 0.663 inch, and therefore a radial dimension of about 0.136 inch, which provides a comfortable load distribution on the skin.

A series of angularly spaced holes 88 through outer region 82 accommodate insertion of saddle section tangs 65. This insertion of tangs 65 within holes 88 permits a fuller rearward movement. cover 78 on tubular base 42 without undesirably increasing the overall profile or diameter of the cover. These holes also facilitate molding.

Inner region 84 is rearwardly recessed or concave, preferably in the generally hemispherical configuration shown, such as with a radius of curvature of about 0.281 inches, but alternately in other shapes within the scope of the invention. The concavity of inner region 84 preferably has a depth of about 0.065 inch and promotes needle exposure when the cover fully retracts. A ring-shaped groove 90 in the rear surface of the forward end face of cover 78 provides a seat for the forward end of compression spring 100.

The generally cylindrical interior surface of tubular main body 80 includes four regions 92 that are spaced ninety degrees apart and which protrude slightly radially inward. At the base of each of regions 92 is a stop tab 94 that juts further radially inward. The radially aligned, forward lip 96 of stop tab 94 serves as a stop abutment for tangs 65 to prevent a forward axial removal of cover 78 from saddle section 46 when assembled thereto. The base of each stop tab 94 is beveled at 98 to provide clearance for the cartridge assembly when cover 78 is fully retracted. Cover 78 is prevented from revolving about saddle section 46, and but for design tolerances does not rotate at all relative thereto, by the sides of stop tabs 94 engaging the side edges of area 60.

The biasing element which shifts cover 78 forward relative to saddle section 46 is preferably a helical compression spring 100 made of metal. Spring 100 includes a forward end 101 which seats within groove 90, and a rearward end 102 which seats within shoulder groove 74. Other spring designs, and other materials of construction, which provide a suitable biasing effect may be substituted in alternate embodiments.

An outer cover or cap 106 of the preferred needle hiding assembly 20 includes a tapered tubular body 108 with a closed end 110 that defines a blind interior hollow 112. Cap 106 is molded from plastic to provide a lightweight part having suitable resiliency for its snap fit attachment to base 42. A circumferential groove 114 within the interior surface of body 108 proximate the rearward end 116 of cap 106 is used to releaseably mount cap 106 to tubular base 42. In the shown embodiment, cap 106 snap locks onto saddle section 46 when cap 106 is moved rearward from the arrangement shown in FIG. 2 such that ribs 59 insert within groove 114. Disconnection of cap 106 from tubular base 42 is accomplished by simply pulling these components apart.

A series of four equally angularly spaced and longitudinally aligned ribs 118 project from the interior surface of body 108 within the forward end of hollow 112. Interior hollow 112 is sized and shaped to accommodate cover main body 80 in closely spaced relationship. The rear face 119 of each rib 118 provides a stop surface against which cover region 82 abuts when cap 106 is placed over cover 78 and then pushed rearward during its connection to the assembly base 42. Ribs 118 are preferably positioned such that when cap 106 is connected to saddle section 46, cover 78 is retracted with tangs 65 inserting within outer region holes 88. In alternate embodiments, in order to move cover 78 rearward during cap mounting, the interior of cap 106 may be otherwise constructed, such as being tapered sufficiently to frictionally engage the lateral facing surface of cover main body 80.

The inventive needle hiding assembly will be further understood in view of the following explanation of aspects of its operation, starting with the injection pen being configured as shown in FIG. 1. At this point, needle hiding assembly 20 has already been installed as a unit by threading base 42 onto the injection pen after inserting the needled end through the base. The presence of cap 106 on needle hiding assembly 20 during mounting limits the possibility of accidental needle sticks associated with inadvertent retraction of an exposed slidable cover.

To prepare the injector for use, cap 106 is pulled to disconnect it from saddle section 46. While cap 106 is being pulled away to the arrangement shown in FIG. 2, spring 100 forces cover 78 to slide forward from a retracted position until lips 96 of stop tabs 94 abut the underside of tangs 65, at which forward position the movement of cover 78 is halted. At this juncture, assembly 20 is arranged as shown in FIGS. 2 and 4 and hides the needle from view, with the forward needle end 36 being surrounded by cover 78 such that its tip is rearward of the dimple defined by inner region 84.

The injector is then ready for injecting use. The injector is moved such that end face outer region 82 contacts the user's skin immediately around the injection site. As the user forces the injector and thereby its fixed needle down toward the injector site, cover 78 begins to slide rearward along saddle section 46 against a returning force generated by spring 100. As the process continues and cover 78 continues to retract, needle tip 36 passes through opening 86 and penetrates the user's skin, all without being seen by the user. The dimpling of the forward surface of cover 78 tends to pucker the user's skin so the skin inserts within the concavity of that surface during cover retraction, which puckering provides stability at the injection site for needle penetration. The depth of penetration by the needle continues to increase until the forward ends of surfaces 60 abut the underside of end face outer region 82. At such time, preferably all of needle 34 forward of hub portion 33 has extended through opening 86 to allow full needle penetration of the user. In alternate embodiments, different portions of the cover, such as the; rear end surface of cover body 80, can abut different portion of the saddle section to halt movement. At the end of the needle insertion, stop tabs 94 are accommodated within openings 64 and tangs 65 extend within holes 88.

After the injector is operated to inject the medicine, the amount of which having been previously selected by the user, the injector is pulled back to withdraw needle 34 from the user's skin. During this pull back, spring 100 biases cover 78 forward to maintain its end face in contact with the user's skin, and to continue to conceal needle 34. When the injector is fully pulled back and cover 78 has moved forward with tangs 65 engaging stop tabs 94, assembly 20 is once again arranged as shown in FIGS. 2 and 4.

To store the injector for its next use, cap 106 is then preferably mounted to the rest of needle hiding assembly 20. After cap 106 is manipulated such that cover 78 is initially inserted into the mouth of interior hollow 112, further movement of the cap and injector together causes cover 78 to penetrate further into hollow 112 without sliding movement of the cover 78 relative to saddle section 46. When cover 78 is fully inserted such that end face outer region 82 abuts rib faces 119, further moving together of the cap and injector shifts needle cover 78 rearward relative to saddle section 46 against an increasing resistance provided by the compression of spring 100. Cap movement continues until groove 114 aligns with ribs 59 and cap 106 snap connects to base 42. At this point, needle hiding assembly 20 is arranged as shown in FIG. 3, in which needle 34 projects forward of cover 78 but is enclosed or fully hidden within cap 106. The components of needle hiding assembly 20 are sized and configured such that cover 78 is at least partially retracted, and preferably as completely retracted as feasible, when stored within cap 106 to limit the profile of the injector.

While this invention has been shown and described as having preferred designs, the present invention may be modified within the spirit and scope of this disclosure. This application is, therefore, intended to cover any variations, uses or adaptations of the invention using its general principles. Further, this application is intended to cover such departures from the present disclosure as commonly known or customary practice in the art to which this invention pertains.

We claim:

1. A needle hiding assembly for a portable injector having a needle forwardly axially protruding from a forward end of the injector, the needle being in communication with a medicine filled cartridge of the injector, the needle hiding assembly comprising:
a base sleeve releaseably connected to the injector;
a needle cover slidably retained on said base sleeve to be axially movable between forward and rearward positions, wherein said needle cover when in said forward position surrounds at least a forward part of the needle, said needle cover including a forward surface that defines a needle accommodating opening through which the needle extends when said needle cover is shifted from said forward position to said rearward position, wherein a length of the needle that projects forward beyond said forward surface is exposed for injection when said needle cover is disposed in said rearward position;
a biasing, element acting between said base sleeve and said needle cover to bias said needle cover from said rearward position toward said forward position; and
a cap releaseably connectable to said base sleeve and defining an interior hollow that accommodates said needle cover, wherein said cap and needle cover are configured such that connecting said cap to said base sleeve shifts said needle cover from said forward position toward said rearward position against a returning force provided by said biasing element.

2. The needle hiding assembly of claim 1 wherein said base sleeve surrounds a forward portion of the injector adjacent the injector forward end.

3. The needle hiding assembly of claim 2 wherein said base sleeve axially extends along at least a majority of a length of the cartridge of the injector.

4. The needle hiding assembly of claim 1 wherein said biasing element comprises a compression spring.

5. The needle hiding assembly of claim 1 wherein said cap includes a radially inwardly protruding stop that abuts said forward surface of said needle cover to shift said needle cover from said forward position toward said rearward position during connection of said cap to said base sleeve.

6. The needle hiding assembly of claim 5 wherein said stop comprises a rearward face of at least one cap rib.

7. The needle hiding assembly of claim 1 wherein said needle cover is slidably retained on said base sleeve by a plurality of outwardly projecting tangs of said base sleeve that engage inwardly projecting stop tabs of said needle cover.

8. The needle hiding assembly of claim 7 wherein said forward surface of said needle cover defines a plurality of tang accommodating holes disposed radially outward of said needle accommodating opening, wherein said tangs fit within said tang accommodating holes when said needle cover is disposed in said rearward position.

9. The needle hiding assembly of claim 1 wherein said forward surface of said needle cover comprises a first region and a second region, wherein said first region encircles said needle accommodating opening and is located between said needle accommodating opening and said second region, and wherein said first region is rearwardly recessed from said second region to define a concavity for increased needle length exposure.

10. The needle hiding assembly of claim 9 wherein said first region of said forward surface is generally hemispherical, and said second region of said forward surface is annular and generally planar.

11. The needle hiding assembly of claim 1 wherein said cap is connectable to said base sleeve by at least one detent in one of said cap and said base sleeve that fits within a circumferential groove in the other of said cap and said base sleeve.

12. The needle hiding assembly of claim 1 wherein said base sleeve comprises at least one window for viewing the medicine within the cartridge.

13. The needle hiding assembly of claim 12 wherein said at least one window is covered by a viewing element having markings for allowing a user to gauge medicine remaining in the cartridge.

14. A needle hiding assembly for an injection pen having a needle forwardly axially protruding from a forward end of the injection pen, the needle being in communication with a medicine filled cartridge of the injection pen, the needle hiding assembly comprising:
a base sleeve releaseably connected to the injection pen;
a needle cover slidably retained on said base sleeve to be axially movable between forward and rearward positions, wherein said needle cover when in said forward position surrounds at least a forward part of the needle, said needle cover including a forward surface that defines a needle accommodating opening through which the needle extends when said needle cover is shifted from said forward position to said rearward position, wherein a length of the needle that projects forward beyond said forward surface is exposed for injection when said needle cover is disposed in said rearward position;
a biasing element acting between said base sleeve and said needle cover to bias said needle cover from said rearward position toward said forward position; and
wherein said forward surface of said needle cover comprises a first region and a second region, wherein said first region encircles said needle accommodating opening and is located between said needle accommodating opening and said second region, and wherein said first region is rearwardly recessed from said second region to define a concavity for increased needle length exposure.

15. The needle hiding assembly of claim 14 wherein said first region of said forward surface is generally hemispherical.

16. The needle hiding assembly of claim 14 wherein said second region of said forward surface is annular and generally planar.

17. The needle hiding assembly of claim 14 wherein said needle cover is slidably retained on said base sleeve by a plurality of outwardly projecting tangs of said base sleeve.

18. The needle hiding assembly of claim 17 wherein said second region of said forward surface of said needle cover defines a plurality of tang accommodating holes disposed radially outward of said needle accommodating opening, wherein said tangs fit within said tang accommodating holes when said needle cover is disposed in said rearward position.
